(19) United States
(12) Patent Application Publication
(10) Pub. No.: US 2024/0355497 A1
(43) Pub. Date: Oct. 24, 2024

(54) METHOD FOR MANUFACTURING PARTICLE BEAM THERAPY SYSTEM AND PARTICLE BEAM THERAPY SYSTEM

(71) Applicants: KABUSHIKI KAISHA TOSHIBA, Tokyo (JP); TOSHIBA ENERGY SYSTEMS & SOLUTIONS CORPORATION, Kawasaki-shi (JP)

(72) Inventors: Hiromichi RYUTO, Yokohama Kanagawa (JP); Atsushi MIYAMOTO, Yokohama Kanagawa (JP); Yutaka HIRATA, Yokohama Kanagawa (JP)

(73) Assignees: KABUSHIKI KAISHA TOSHIBA, Tokyo (JP); TOSHIBA ENERGY SYSTEMS & SOLUTIONS CORPORATION, Kawasaki-shi (JP)

(21) Appl. No.: 18/758,983

(22) Filed: Jun. 28, 2024

Related U.S. Application Data
(63) Continuation of application No. PCT/JP2023/000833, filed on Jan. 13, 2023.

(30) Foreign Application Priority Data
Feb. 4, 2022 (JP) 2022-016263
Jan. 10, 2023 (JP) 2023-001529

(51) Int. Cl. G21K 5/04; G21K 5/10

(52) U.S. Cl. CPC G21K 5/04; G21K 5/10

(57) ABSTRACT

According to one embodiment, a method for manufacturing a particle beam therapy system that comprising steps of: designing a main transport line such that a phase lead of a beta function representing a betatron oscillation of a charged particles passing through the main transport line from a first connection point to a second connection point is an integer multiple of π; and setting a beam shape such that respective beam optical parameters match at each boundary between a rotating portion and a fixed portion of each of a plurality of rotating gantries.

### CROSS-REFERENCE TO RELATED APPLICATIONS

This application is a Continuation Application of No. PCT/JP2023/000833, filed on Jan. 13, 2023, and the PCT application is based upon and claims the benefit of priority from Japanese Patent Applications No. 2022-016263, and No. 2023-001529, filed on Feb. 4, 2022, and Jan. 10, 2023, the entire contents of which are incorporated herein by reference.

### TECHNICAL FIELD

Embodiments of the present invention relate to manufacturing techniques for a particle beam therapy system.

### BACKGROUND

In case of introducing particle beams into a plurality of treatment rooms in a particle beam therapy system, lines of transporting the particle beams are extended, branched, and bent so as to match layout of these treatment rooms. However, distribution of charged particles in the beam passing through the lines is not constant, its cross-sectional shape changes along the lines of transporting the particle beams, causing oscillation with a constant period called a betatron oscillation. Thus, the lines of transporting the particle beams are required to have design specifications that correspond to the cross-sectional shape of the beam passing through it. As the lines are extended longer or the number of line branches increases, the time required for line design and on-site adjustment increases exponentially, which increases its construction period and construction cost.

For this reason, there is a known technique to construct a beam transport line by combining segments, each of which has constituent components in common. However, with this technique alone, it is difficult to freely construct the beam transport line depending on the layout of the plurality of treatment rooms or the size of the land at the construction site.

In another known technique, in the beam transport line, the phase difference of the betatron oscillation of a charged particle beam between a first branch point and a second branch point is set to an integer multiple of π and Twiss parameters are set to be the same between respective branch points. This technique is for designing a treatment room (i.e., fixed room) in which an irradiation port is fixed and can sufficiently facilitate the design of the beam transport line in the case of installing an additional fixed room. However, in the case of designing a treatment room (i.e., rotating gantry room) in which the irradiation port is movable by a rotating gantry, this technique cannot sufficiently facilitate the design of the beam transport line.

### PRIOR ART DOCUMENT

### Patent Document

[Patent Document 1] JP 2017-029235 A

[Patent Document 2] JP 2017-020813 A

### SUMMARY

### Problem to be Solved by Invention

Regarding a particle beam therapy system provided with a plurality of treatment rooms in respective rotating gantries, the present invention aims to provide a technique that is for manufacturing such a particle beam therapy system and can facilitate the design of its beam transport line and contribute to reduction in its construction period and construction cost in the case of installing an additional rotating gantry.

### BRIEF DESCRIPTION OF DRAWINGS

FIG. 1 is a plan view illustrating a particle beam therapy system.

### DETAILED DESCRIPTION

In one embodiment of the present invention, a method for manufacturing a particle beam therapy system that comprises: a circular accelerator configured to accelerate charged particles; a beam transport line configured to guide the charged particles accelerated by the circular accelerator to a plurality of treatment rooms; and a plurality of rotating gantries, each of which can change an irradiation direction of the charged particles guided by the beam transport line with respect to a patient and has the respective treatment rooms inside, wherein the beam transport line includes a main transport line extending from the circular accelerator and a plurality of sub transport lines extending from the main transport line to the respective treatment rooms, the plurality of sub transport lines include one sub transport line and another sub transport line, the one sub transport line is connected to a first connection point of the main transport line, and the another sub transport line is connected to a second connection point of the main transport line, the second connection point being different from the first connection point, the method comprising steps of: designing the main transport line such that a phase lead of a beta function representing a betatron oscillation of the charged particles passing through the main transport line from the first connection point to the second connection point is an integer multiple of π; and setting a beam shape such that respective beam optical parameters match at each boundary between a rotating portion and a fixed portion of each of the plurality of rotating gantries.

According to embodiments of the present invention, it is possible to provide a technique that is for manufacturing such a particle beam therapy system and can facilitate the design of its beam transport line and contribute to reduction in its construction period and construction cost in the case of installing an additional rotating gantry.

Hereinbelow, embodiments of a particle beam therapy system and a method for manufacturing the particle beam therapy system will be described in detail by referring to the accompanying drawings.

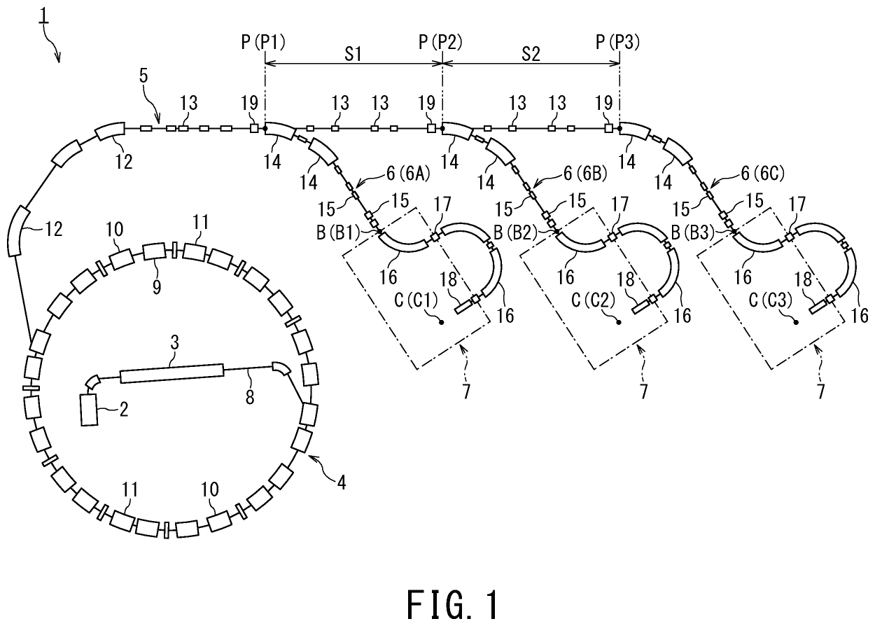

The reference sign 1 in FIG. 1 denotes a particle beam therapy system according to the present embodiment. This particle beam therapy system 1 is a so-called particle-beam cancer treatment apparatus that performs treatment by irradiating a patient's diseased tissue such as a carcinomatous lesion with a particle beam such as carbon ion as therapeutic radioactive ray.

A radiation therapy technique with the use of the particle beam therapy system 1 is also referred to as a heavy ion beam cancer treatment technique. This technique is said to be able to damage the cancerous lesion (i.e., focus of disease) and minimize the damage to normal cells by pinpointing the cancerous lesion with carbon ions. Note that the particle beam is defined as radioactive ray heavier than electron, and include proton beam and heavy ion beam, for example. Of this, heavy ion beam is defined as radioactive ray heavier than helium atom.

As compared with the conventional cancer treatment using X-ray, gamma ray, or proton beam, the cancer treatment using heavy ion beams has characteristics that: (i) the ability to kill the cancerous lesion is higher; and (ii) the radiation dose is weak on the surface of the body of the patient so as to peak at the cancerous lesion. Thus, the number of irradiations and side effects can be reduced, and the treatment period can be shortened.

The particle beam loses its kinetic energy at the time of passing through the body of the patient so as to decrease its velocity and receive a resistance that is approximately inversely proportional to the square of the velocity and stops rapidly when it decreases to a certain velocity. The stopping point of the particle beam is referred to as the Bragg peak at which high energy is emitted. The particle beam therapy system 1 matches this Bragg peak with the position of the lesion tissue (i.e., affected part) of the patient, and thus, can kill only the lesion tissue while suppressing the damage to normal tissues.

The particle beam therapy system 1 includes a beam generator 2, a linear accelerator 3, a circular accelerator 4, a main transport line 5, a plurality of sub transport lines 6, and a plurality of rotating gantries 7. Note that the main transport line 5 and the sub transport lines 6 constitute a beam transport line.

The beam generator 2 has an ion source of carbon ions, which are charged particles, and uses these carbon ions to generate a particle beam. The linear accelerator 3 has a linear shape in a plan view and accelerates the ions generated by the beam generator 2 into a particle beam. The linear accelerator 3 introduces this particle beam into the circular accelerator 4.

The circular accelerator 4 has a ring shape in a plan view, and further accelerates the particle beam. The particle beam is accelerated to approximately 70% of the speed of light while orbiting the circular accelerator 4 approximately one million times. The particle beam accelerated by the circular accelerator 4 is transported to a selected rotating gantry 7 through the main transport line 5 and the corresponding sub transport line 6. A patient to be irradiated with the particle beam is placed inside the selected rotating gantry 7. The inside of each rotating gantry 7 is a treatment room (i.e., rotating gantry room).

The beam generator 2, the linear accelerator 3, the circular accelerator 4, the main transport line 5, and the sub transport lines 6 are provided with an integrally extended vacuum duct 8 (i.e., beam pipe), inside of which is vacuumized. The particle beam passes the inside of the vacuum duct 8. This vacuum duct 8 forms a transport path that guides the particle beam from the beam generator 2 to the respective rotating gantries 7. In other words, the vacuum duct 8 is a closed continuous space with a sufficient degree of vacuum to allow the particle beam to pass through.

The circular accelerator 4 includes a high-frequency acceleration cavity 9, bending electromagnets 10, and convergence electromagnets 11. The high-frequency acceleration cavity 9 accelerates carbon ions by controlling respective frequencies of an acceleration electric field and a magnetic field.

The bending electromagnets 10 and the convergence electromagnets 11 are electromagnets configured to generate magnetic fields that form a transport path of the particle beam and are arranged so as to surround the outer periphery of the vacuum duct 8. The bending electromagnets 10 change the traveling direction of the particle beam along the vacuum duct 8. In addition, the convergence electromagnets 11 control convergence and divergence of the particle beam. The convergence electromagnets 11 are composed of quadrupole electromagnets or hexapole electromagnets.

The main transport line 5 includes bending electromagnets 12 and convergence electromagnets 13. The main transport line 5 extends from the circular accelerator 4. The plurality of sub transport lines 6 are connected to the linear portion of the main transport line 5.

Each of the sub transport lines 6 includes bending electromagnets 14 and convergence electromagnets 15. In the present embodiment, three sub transport lines 6 are connected to one main transport line 5. Each sub transport line 6 extends to the rotating gantry 7.

In other words, the beam transport line composed of the main transport line 5 and the plurality of sub transport lines 6 guides the particle beam accelerated by the circular accelerator 4 to the respective treatment rooms inside the rotating gantries 7.

Although detailed illustrations are omitted, each rotating gantry 7 is a cylindrical apparatus. Each rotating gantry 7 is disposed in such a manner that its cylindrical axis is aligned in the horizontal direction. Each rotating gantry 7 is rotatable around this horizontal axis.

Each rotating gantry 7 is supported by a framework of a building (not shown) constituting the treatment facility where the particle beam therapy system 1 is installed. For example, end rings (not shown) are fixed to the front edge and the rear edge of each rotating gantry 7. Below these end rings of each rotating gantry 7, a rotary drive unit (not shown) configured to rotatably support the end rings and provided with a drive motor is installed. Each rotary drive unit is supported by the framework. The driving force of each rotary drive unit is applied to the rotating gantry 7 through the end rings, and thereby each rotating gantry 7 is rotated around the horizontal axis.

Each rotating gantry 7 also includes bending electromagnets 16, convergence electromagnets 17, and an irradiation nozzle 18. The bending electromagnets 16, the convergence electromagnets 17, and the irradiation nozzle 18 are supported by the rotating gantry 7 and can rotate together with the rotating gantry 7.

Note that the bending electromagnets 10, 12, 14, 16 and the convergence electromagnets 11, 13, 15, 17 may be configured as superconducting electromagnets.

Each rotating gantry 7 is connected to the vacuum duct 8 that continues from the sub transport line 6. The vacuum duct 8 is first guided from the end of the rotating gantry 7 to the inside along its horizontal axis. The vacuum duct 8 once extends outward from the outer circumferential surface of the rotating gantry 7, and then extends again towards the inside of the rotating gantry 7. The irradiation nozzle 18, in which the tip of the vacuum duct 8 is placed, extends to a position close to the patient.

Of the vacuum duct 8, the portion along the horizontal axis of the rotating gantry 7 is provided with a predetermined rotation mechanism (not shown). The portion of the vacuum duct 8 outside this rotating mechanism is in a stationary state, and the portion of the vacuum duct 8 inside this rotating mechanism is configured to rotate together with the rotation of the rotating gantry 7.

The irradiation nozzle 18 is provided at the tip of the vacuum duct 8 and irradiates the patient with the particle beam guided by the bending electromagnets 16 and the convergence electromagnets 17. The irradiation nozzle 18 is fixed to the inner circumferential surface of the rotating gantry 7. Note that the particle beam is radiated from the irradiation nozzle 18 in the direction perpendicular to the horizontal axis.

The patient is placed on a treatment table (not shown) provided in the treatment room inside each rotating gantry 7. This treatment table can be moved in the state where the patient is placed thereon. The patient can be moved to the irradiation position of the particle beam by moving this treatment table, and thereby positioning can be performed. Thus, the diseased tissue of the patient can be irradiated with the particle beam with optimal precision.

In each treatment room, an isocenter C is set as the position where the particle beam is most concentratedly radiated. Before start of treatment, a lesion site of the patient is located at the isocenter C by moving the treatment table while a location of the lesion site is being checked by using X-ray images, for example. The beam shape (i.e., beam profile) is set in such a manner that a node of a betatron oscillation is located at this isocenter C. In other words, the beam optical parameters that determine the beam profile are set.

The beam optical parameters are expressed by at least one of an alpha function, a beta function, a gamma function, an emittance, and a dispersion. Each of the alpha function, the beta function, and the gamma function is a parameter indicative of a beam trajectory in an equation that represents the beam trajectory as an equation of simple harmonic oscillation. In addition, the emittance is a parameter that indicates the spread of the beam. Further, the dispersion is also called a dispersion function and is a parameter that represents the relationship between the beam momentum and the beam position.

The patient is positioned on the horizontal axis, and the irradiation nozzle 18 can be rotated around the stationary patient by rotating the rotating gantry 7. For example, the irradiation nozzle 18 can be rotated by a maximum of 180° to one side and to the opposite side in the circumferential direction of the rotating gantry 7, centering on the patient (i.e., around the horizontal axis), and thus, the rotating gantry 7 can be rotated to any angle within a total range of 360°. Accordingly, the particle beam can be radiated from any direction around the patient. In other words, the rotating gantry 7 is an apparatus configured to be able to change the irradiation direction of the particle beam guided by the sub transport line 6 with respect to the patient, and thus, can accurately irradiate the lesion site with the particle beam from the optimal direction while reducing the burden on the patient.

Next, a description will be given of the method for manufacturing the particle beam therapy system 1 according to the present embodiment.

In the present embodiment, the plurality of sub transport lines 6 include a plurality of sub transport lines 6A, 6B, 6C. In the linear portion of the main transport line 5, one (first) sub transport line 6A is connected and branched to a first point P1. In addition, another (second) sub transport line 6B is connected and branched to a second point P2. Furthermore, another (third) sub transport line 6C is connected and extended to a third point P3.

It is assumed that the first point P1 is a first connection point and the second point P2 different from the the first connection point is a second connection point. Under this assumption, the main transport line 5 is designed in such a manner that the phase lead (i.e., phase advance) of the beta function representing the betatron oscillation of the charged particles passing through the main transport line 5 from the first point P1 (i.e., the first connection point) to the second point P2 (i.e., the second connection point) is an integer multiple of π. This design can save the labor of beam adjustment for each connection point P and can commonalize the beam adjustment for each connection point P.

Also under the assumption that the second point P2 is the first connection point and the third point P3 different from this is the second connection point, the main transport line 5 is similarly designed in such a manner that the phase lead from the second point P2 (i.e., the first connection point) to the third point P3 (i.e., the second connection point) becomes an integer multiple of π.

For example, the beam shape depends on the betatron oscillation around the central orbit, and thus can be expressed as a beta function. As can be seen from an expression of a transport matrix expressed by the parameters of the betatron oscillation, changes in the beam shape are expressed by trigonometric functions. Hence, when the phase lead from one point to another point is an integer multiple of π, the respective beam shapes at these two points become the same.

The betatron oscillation is determined by the magnetic field distribution. Thus, once the strength and arrangement of the convergence electromagnets 13 are determined at the time of designing the main transport line 5, the beam shape at a predetermined point on the main transport line 5 is uniquely determined.

For example, when the main transport line 5 and the first sub transport line 6A are connected at the first point P1 (i.e., the first connection point), the configuration of the rotating gantry 7 and the sub transport line 6A from the first point P1 to the position of the isocenter C1 is determined in advance. Further, it is designed in such a manner that the phase lead from the first point P1 to the second point P2 (i.e., the second connection point) is an integral multiple of π. Moreover, it is designed in such a manner that the phase lead from the second point P2 (i.e., the first connection point) to the third point P3 (i.e., the second connection point) is also an integer multiple of π. In this manner, similarly to the configuration of the first sub transport line 6A extending from the first point P1 and the rotating gantry 7 connected thereto, the configurations of the second and third sub transport lines 6B and 6C and the respective rotating gantries 7 can be made common. Furthermore, similar beam shapes can be achieved at the isocenters C1, C2, and C3 in the respective treatment rooms.

In addition, the number of treatment rooms (i.e., rotating gantry rooms) can be increased by repeating the configuration from the first connection point to the second connection point in the main transport line 5 without having to calculate a new trajectory of the particle beam.

For example, the layout of treatment rooms may differ depending on the treatment facility. In the conventional technology, it is necessary to calculate the particle beam trajectory (i.e., lattice calculation) for each treatment facility and design it in such a manner that the beam shape at the position (i.e., the isocenter C) where the particle beam is radiated onto the patient is constant.

In the present embodiment, even if the number of the sub transport lines 6 is increased, the beam shape at each connection point P connected with the main transport line 5 remains the same, and thus, the design of the downstream configuration of each sub transport line 6 is facilitated. In other words, the configuration on the downstream side of the connection point P can be shared or commonalized. It can also contribute to suppression or reduction in the construction period and construction cost of the particle beam therapy system 1. Furthermore, the beam transport line can be freely constructed depending on the situation, such as the layout of the plurality of treatment rooms and the size of the land at the construction site.

The beam optical parameters of the present embodiment include the Twiss parameter. A designer of the particle beam therapy system 1 designs the main transport line 5 in such a manner that the Twiss parameters of the particle beam are the same between the respective connection points P of the first point P1, the second point P2, and the third point P3 in the main transport line 5. In this manner, the beam shapes become the same at the respective connection points P, and thereby, the design on the downstream side of each connection point P can be shared or commonalized.

In addition, the strength and arrangement of at least the convergence electromagnets 13 provided on the main transport line 5 are adjusted in such a manner that the phase lead from the first connection point to the second connection point becomes an integral multiple of π. In this manner, the phase lead of the beta function can be adjusted by adjusting the convergence electromagnets 13. For example, the convergence electromagnets 13 between the first connection point and the second connection point are adjusted. Additionally, or alternatively, the convergence electromagnets 13 excluding the ones provided between the first connection point and the second connection point may be adjusted. Moreover, the strength and arrangement of the bending electromagnets 12 provided on the main transport line 5 may be adjusted.

In the present embodiment, at least three sub transport lines 6A, 6B, and 6C are connected to one main transport line 5, and there are at least two sections corresponding to the range from the first connection point to the second connection point. For example, a first section S1 from the first point P1 to the second point P2 and a second section S2 from the second point P2 to the third point P3 are provided.

The designer designs the particle beam therapy system 1 in such a manner that at least the convergence electromagnets 13 provided in the first section S1 and the second section S2 are the same in terms of strength and arrangement. Additionally, or alternatively, the first section S1 and the second section S2 are designed to have the same length. In this manner, the respective beam shapes at the first connection point and the second connection point can be made the same.

Although the convergence electromagnets 13 provided in the first section S1 are the same in strength and arrangement as the convergence electromagnets 13 provided in the second section S2 in the present embodiment, another configuration may be adopted. For example, the convergence electromagnets 13 provided in the first section S1 may be different in strength and arrangement from the convergence electromagnets 13 provided in the second section S2.

Although the first section S1 and the second section S2 have the same length in the present embodiment, another aspect may be adopted. For example, the first section S1 may be different in length from the second section S2.

In the present embodiment, a plurality of beam monitors 19 (i.e., screen monitors) are provided at the respective connection points P of the first point P1, the second point P2, and the third point P3 in the main transport line 5, i.e., at the respective positions corresponding to the first connection point and the second connection point. For example, the respective beam monitors 19 are provided on the upstream side of each connection point P. In other words, the respective beam monitors 19 are provided near the entrance side of the bending electromagnets 14 on the entrance side of each sub transport line 6.

Note that the respective beam monitors 19 may be provided on the downstream side of each connection point P. In other words, the respective beam monitors 19 may be provided near the exit side of the bending electromagnets 14 on the entrance side of each sub transport line 6.

Note that the distances from the respective connection points P to the respective beam monitors 19 are the same. The respective beam monitors 19 measure the beam shape of the charged particles. In this manner, adjustment of the beam shape of the charged particles at each connection point P is facilitated. For example, this configuration can achieve a comparison as to whether the beam shapes at the respective connection points P are the same or not.

Note that measurement of the beam shape using the respective beam monitors 19 may be performed during the manufacture of the particle beam therapy system 1 or may be performed during operation of the particle beam therapy system 1.

In the present embodiment, even if the irradiation direction is changed by the rotating gantry 7, adjustment of the beam shape of the charged particles to be radiated to the patient is facilitated. For example, in the rotating gantry 7, the betatron oscillation is analyzed by using the connection point P as a reference, and the beam shape is set in such a manner that the node of the betatron oscillation is located at the boundary B between the rotating portion and the fixed portion. Although precise calculation is required in the conventional technology for keeping the beam shape at the isocenter C constant even in the case of rotating the rotating gantry 7 and this calculation requires labor and cost, the present embodiment can solve such a problem.

In addition, the designer of the particle beam therapy system 1 sets the beam shape in such a manner that the respective beam optical parameters match at each the boundaries B1, B2, and B3 between the rotating portion and the fixed portion of the respective rotating gantries 7. Under such a design, for example, the construction period and construction cost can be suppressed or reduced in the case of initially constructing two rotating gantries 7 and then adding the remaining rotating gantry 7.

For example, when the beam shape is set in such a manner that the beam optical parameters are the same between the respective boundaries B1 and B2, the length from the first point P1 to the boundary B1 may be different from the length from the second point P2 to the boundary B2. In this manner, even if the third point P3 is added later and the boundary B3 is extended from the third point P3 and the new rotating gantry 7 is added beyond that, the design of the main transport line 5 and the sub transport lines 6 can be facilitated.

In addition, the designer of the particle beam therapy system 1 sets the beam shape in such a manner that the beam optical parameters at the isocenter C become the same as the beam optical parameters at the boundary B between the rotating portion and the fixed portion of the rotating gantry 7. In this manner, the beam shape at the isocenter C become the same no matter where the angle of the rotating gantry 7 is positioned.

There are several ways for setting the beam shape in the present embodiment. The designer of the particle beam therapy system 1 sets the beam shape in such a manner that conditions under at least one of a symmetric beam method, a round beam method, and a rotator method are satisfied at each boundary B1, B2, B3. Either method can facilitate the design of the main transport line 5 and the sub transport lines 6.

When these methods are applied, the size and shape of the beam spot does not change even if the rotating gantry 7 rotates. Furthermore, in the bending electromagnets 16 and the convergence electromagnets 17 included in each rotating gantry 7, the above-described configuration can ensure that the trajectory of the particle beam does not change even if the angle of the rotating gantry 7 changes.

The symmetrical beam method is a method in which the beam shape is set to be rotationally symmetrical with respect to the rotational direction of the rotating gantry 7 at the entrance (i.e., the boundary B) of the rotating gantry 7. For example, when the traveling direction of the particle beam is assumed to be the Z-axis, the beam shape is set so as to form the same phase space ellipse on the X-axis (i.e., the horizontal axis) and the Y-axis (i.e., the vertical axis). This symmetric beam method is a method in which the beam optical parameters and the emittance of the X-axis and the Y-axis are matched at the entrance of the rotating gantry 7 and the dispersion is set to zero.

The round beam method is a method in which the phase lead of the beam optical system (from the boundary B to the isocenter C) composed of the bending electromagnets 16 and the convergence electromagnets 17 provided in each rotating gantry 7 is set to an integral multiple of π and the dispersion at the entrance (i.e., the boundary B) is set to zero. For example, when a round beam is made incident on the entrance of the rotating gantry 7, a round beam is also obtained at the isocenter C.

The rotator method is a method in which a rotator (not shown) configured as a portion to be rotated by half the rotation angle of the rotating gantry 7 is provided on the upstream side of the entrance (i.e., the boundary B) of each rotating gantry 7. This rotator rotates the convergence electromagnets 15 located immediately in front of the upstream side of the rotating gantry 7 in the sub transport line 6 by half the rotation angle of the rotating gantry 7, for example. When the traveling direction of the particle beam is assumed to be the Z-axis, this rotator method is a method in which the phase lead of the X-axis (i.e., the horizontal axis) from the entrance (i.e., the upstream end) to the exit (i.e., the downstream end) of the rotator is set to 2π and the phase lead of the Y-axis (i.e., the vertical axis) is set to π.

In addition, the designer of the particle beam therapy system 1 sets the beam shape in such a manner that the Twiss parameters of the X-axis and the Y-axis are equal at the boundary B under the assumption that the traveling direction of the particle beam is the Z-axis. In this manner, even if the rotation angle of the rotating gantry 7 changes, the beam shape can be maintained constant at the isocenter C (i.e., the irradiation position).

Further, the designer of the particle beam therapy system 1 sets the respective Twiss parameters of the first point P1, the second point P2, and the third point P3 in such a manner that the Twiss parameters of the X-axis and the Y-axis are equal at each boundary B1, B2, and B3. In this manner, the construction period and construction cost in the case of installing an additional rotating gantry 7 can be suppressed or reduced.

The designer designs the particle beam therapy system 1 in such a manner that at least one of the alpha function, the beta function, the gamma function, the emittance, and the dispersion representing the beam optical parameters is matched at respective boundaries B1, B2, and B3.

Additionally, the designer of the particle beam therapy system 1 adjusts the strength and arrangement of the bending electromagnets 12, 14 and the convergence electromagnets 13, 15 provided on the main transport line 5 and the sub transport lines 6 in such a manner that the above-described beam optical parameters match at the respective boundaries B1, B2, B3. In this manner, the beam optical parameters can be adjusted by adjusting the strength and arrangement of the bending electromagnets 12, 14 and the convergence electromagnets 13, 15.

Although a description has been given of the configuration in which every treatment room is provided in one rotating gantry 7 in the present embodiment, another configuration may be adopted. For example, instead of providing the rotating gantry 7, a treatment room in which the irradiation nozzle 18 is fixedly disposed may be provided.

Although the connection points P to which the respective sub transport lines 6 are connected are provided on the linear portion of the main transport line 5 in the present embodiment, another configuration may be adopted. For example, each connection point P may be provided in a curved portion of the main transport line 5. In addition, it may be configured such that the first section S1 is straight and the second section S2 is curved.

Although the present embodiment illustrates the rotating gantry 7 configured as a fully rotating type that can be rotated by a maximum of 180° both in one direction in the circumferential direction and in the opposite direction in the circumferential direction and thus can be rotated to any angle within a total range of 360°, another configuration of the rotating gantry 7 may also be used. For example, the present embodiment may be applied to a half-rotation type gantry, i.e., so-called half gantry that can be rotated by a maximum of 90° both in one direction in the circumferential direction and in the opposite direction in the circumferential direction and thus can be rotated to any angle within a total range of 180°. The half gantry refers to an apparatus, rotation range of which is less than two-thirds of the entire circumference (240° or less).

Although a description has been given of the particle beam using carbons in the present embodiment, other aspects may also be adopted. For example, a particle beam using helium, oxygen, or neon may also be used.

According to the above-described embodiments, in the particle beam therapy system 1 provided with the plurality of treatment rooms in the respective rotating gantries 7, the design of the beam transport line is facilitated, which can contribute to suppression or reduction in its construction period and construction cost in the case of installing an additional rotating gantry.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope 10 and spirit of the inventions. The articles “the”, “a” and “an” are not necessarily limited to mean only one, but rather are inclusive and open ended so as to include, optionally, multiple such elements.

1. A method for manufacturing a particle beam therapy system that comprises:
a circular accelerator configured to accelerate charged particles;
a beam transport line configured to guide the charged particles accelerated by the circular accelerator to a plurality of treatment rooms; and
a plurality of rotating gantries, each of which can change an irradiation direction of the charged particles guided by the beam transport line with respect to a patient and has the respective treatment rooms inside,
wherein the beam transport line includes a main transport line extending from the circular accelerator and a plurality of sub transport lines extending from the main transport line to the respective treatment rooms,
the plurality of sub transport lines include one sub transport line and another sub transport line,
the one sub transport line is connected to a first connection point of the main transport line, and
the another sub transport line is connected to a second connection point of the main transport line, the second connection point being different from the first connection point,
the method comprising steps of:
designing the main transport line such that a phase lead of a beta function representing a betatron oscillation of the charged particles passing through the main transport line from the first connection point to the second connection point is an integer multiple of π; and
setting a beam shape such that respective beam optical parameters match at each boundary between a rotating portion and a fixed portion of each of the plurality of rotating gantries.

2. The method for manufacturing a particle beam therapy system according to claim 1, wherein the beam shape is set such that the respective beam optical parameters match at the boundary and a position in each of the treatment rooms to be most concentratedly irradiated with the charged particles.

3. The method for manufacturing a particle beam therapy system according to claim 1, wherein the beam shape is set such that conditions under at least one of a symmetric beam method, a round beam method, and a rotator method are satisfied at each boundary.

4. The method for manufacturing a particle beam therapy system according to claim 1, wherein:
the beam optical parameters include a Twiss parameter; and
the beam shape is set such that Twiss parameters of an X-axis and a Y-axis become equal at the boundary under an assumption that a traveling direction of the charged particles is a Z-axis.

5. The method for manufacturing a particle beam therapy system according to claim 4, wherein the Twiss parameters of the first connection point and the second connection point are set such that the Twiss parameters of the X-axis and the Y-axis are equal at each boundary.

6. The method for manufacturing a particle beam therapy system according to claim 1, wherein:
the beam optical parameters are expressed by at least one of an alpha function, a beta function, a gamma function, an emittance, and a dispersion; and
at least one of the alpha function, the beta function, the gamma function, the emittance, and the dispersion is matched at each boundary.

7. The method for manufacturing a particle beam therapy system according to claim 1, further comprising a step of adjusting strength and arrangement of a bending electromagnet and a convergence electromagnet provided in the beam transport line such that the respective beam optical parameters match at each boundary.

8. A particle beam therapy system comprising:
a circular accelerator configured to accelerate charged particles;
a beam transport line configured to guide the charged particles accelerated by the circular accelerator to a plurality of treatment rooms; and
a plurality of rotating gantries, each of which can change an irradiation direction of the charged particles guided by the beam transport line with respect to a patient and has the respective treatment rooms inside,
wherein the beam transport line includes a main transport line extending from the circular accelerator and a plurality of sub transport lines extending from the main transport line to the respective treatment rooms,
the plurality of sub transport lines include one sub transport line and another sub transport line,
the one sub transport line is connected to a first connection point of the main transport line,
the another sub transport line is connected to a second connection point of the main transport line, the second connection point being different from the first connection point,
the main transport line is designed such that a phase lead of a beta function representing a betatron oscillation of the charged particles passing through the main transport line from the first connection point to the second connection point is an integer multiple of π, and
a beam shape is set such that respective beam optical parameters match at each boundary between a rotating portion and a fixed portion of each of the plurality of rotating gantries.
